(19) United States
(12) Patent Application Publication
(10) Pub. No.: US 2022/0381130 A1
(43) Pub. Date: Dec. 1, 2022

(54) FORMATION AND RESERVOIR ROCK MODELING USING SYMBOLIC REGRESSION

(71) Applicant: Halliburton Energy Services, Inc., Houston, TX (US)

(72) Inventors: Songhua Chen, Houston, TX (US); Wei Shao, Houston, TX (US)

(21) Appl. No.: 17/735,727

(22) Filed: May 3, 2022

Related U.S. Application Data
(60) Provisional application No. 63/193,934, filed on May 27, 2021.

(51) Int. Cl. E21B 44/00; G01N 33/24; G01V 11/00; E21B 49/00; G06F 30/10

(52) U.S. Cl. CPC E21B 44/00; G01N 33/24; G01V 11/00; E21B 49/00; G06F 30/10; E21B 2200/20; E21B 2200/22

(57) ABSTRACT

System and methods of petrophysical modeling are disclosed. Training data for modeling a reservoir formation surrounding a wellbore drilled within the reservoir formation is received via a network from one or more data sources. A machine learning model is trained using symbolic regression to determine a formation model representing the reservoir formation, based on the training data received from the data source(s). At least one property of the reservoir formation is estimated, based on the formation model. A downhole operation is performed along the wellbore within the reservoir formation, based on the at least one estimated property.

### CROSS-REFERENCE TO RELATED APPLICATIONS

This application claims priority to U.S. Provisional Application No. 63/193,934, filed on May 27, 2021, the benefit of which is claimed and the disclosure of which is incorporated herein by reference in its entirety.

### FIELD OF THE DISCLOSURE

The present disclosure relates generally to petrophysical modeling and particularly, to using symbolic regression to model petrophysical rock properties and fluid saturations of a hydrocarbon bearing formation.

### BACKGROUND

Development of a petrophysical interpretation model for a reservoir rock formation is often starts with laboratory analysis of core samples obtained from the formation. The results from such a core analysis may be used to determine different sets of petrophysical parameters associated with the formation. For example, routine core analysis (RCA) may be carried out on a core sample to measure porosity, grain density, horizontal permeability, fluid saturation, a lithologic description, and/or the like. The RCA may be combined with well logging measurements including, but not limited to, natural gamma ray and neutron logs. Further, special core analysis (SCA) may be employed to determine additional formation parameters, such as measurements of wettability, electrical properties, two-phase flow properties, capillary pressure, and/or the like. The parameters obtained from such core analysis may then be used to estimate rock properties and fluid saturations of the formation. Petrophysicists generally use a set of petrophysical equations to develop a model of the formation that can then be used to perform this estimation. Such equations may include, for example, Archie equations, which tend to work well for certain types of rocks (referred to as “Archie rocks”), such as clean sandstones, may be relatively straightforward to estimate. However, the same is not true for various shaly sands, source rocks containing shale oil and shale gas, or carbonates (or “non-Archie rocks”). Petrophysicists may have difficulty using Archie equations to model formation parameters and estimate properties rock properties, such as saturation and rock formation factor, for a reservoir formation having several formation beddings with different lithology or mineralogy characteristics. In such cases, a labor intensive and often error-prone procedure may be required to identify appropriate sets of Archie parameters needed to effectively model different lithofacies and/or electrical facies of the formation.

### BRIEF DESCRIPTION OF THE DRAWINGS

FIG. 1 is a diagram of an illustrative drilling system in which embodiments of the present disclosure may be implemented.

FIG. 2 is a flowchart of an illustrative process for estimating a property of a reservoir formation, in accordance with embodiments of the present disclosure.

FIG. 3 is a block diagram of an illustrative system in which embodiments of the present disclosure may be implemented.

FIG. 4 is a flowchart of an illustrative process for determining a property of a reservoir formation using a symbolic regression model, in accordance with embodiments of the is present disclosure.

FIG. 5 is a flowchart of an illustrative process for training a symbolic regression model, in accordance with embodiments of the present disclosure.

FIG. 6 is a table of an illustrative dataset for training a symbolic regression model, in accordance with embodiments of the present disclosure.

FIG. 7 is a table of controlling factors and logging measurements associated with a property of a reservoir formation, in accordance with embodiments of the present disclosure.

FIG. 8 is a block diagram of an illustrative computer system in which embodiments of the present disclosure may be implemented.

### DESCRIPTION OF ILLUSTRATIVE EMBODIMENTS

Embodiments of the present disclosure relate to petrophysical modeling. More specifically, the present disclosure relates to using symbolic regression and machine learning to develop a petrophysical model of a formation that accurately represents the underlying physics of the formation, as captured by a variety of measurements and/or instruments used to obtain the measurements. While the present disclosure is described herein with reference to illustrative embodiments for particular applications, it should be understood that embodiments are not limited thereto. Other embodiments are possible, and modifications can be made to the embodiments within the spirit and scope of the teachings herein and additional fields in which the embodiments would be of significant utility. Further, when a particular feature, structure, or characteristic is described in connection with an embodiment, it is submitted that it is within the knowledge of one skilled in the relevant art to implement such feature, structure, or characteristic in connection with other embodiments whether or not explicitly described.

It would also be apparent to one of skill in the relevant art that the embodiments, as described herein, can be implemented in many different embodiments of software, hardware, firmware, and/or the entities illustrated in the figures. Any actual software code with the specialized control of hardware to implement embodiments is not limiting of the detailed description. Thus, the operational behavior of embodiments will be described with the understanding that modifications and variations of the embodiments are possible, given the level of detail presented herein.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment.

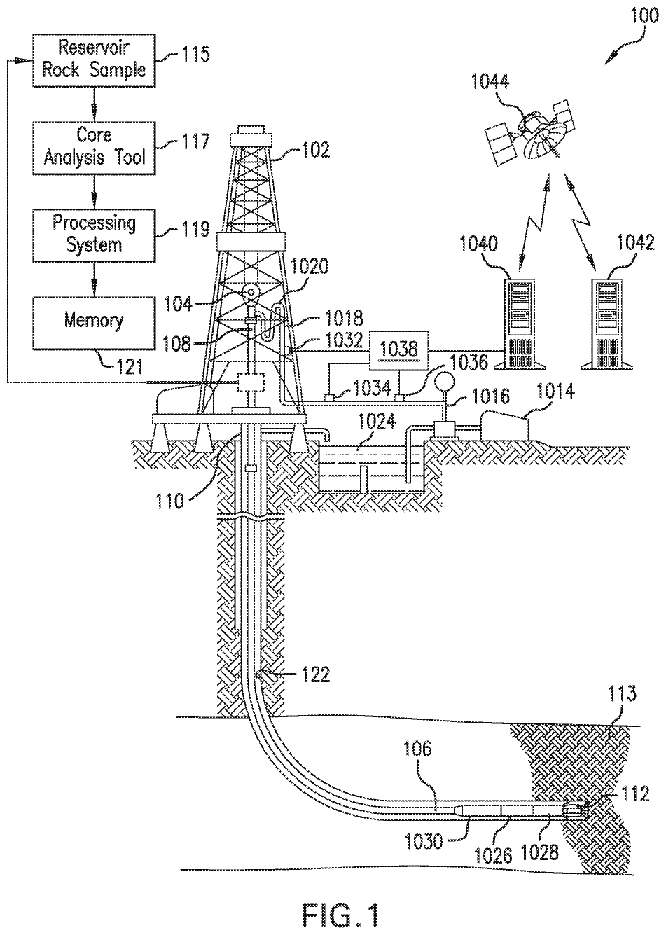

Illustrative embodiments and related methodologies of the present disclosure are described below in reference to FIGS. 1-8 as they might be employed in, for example, a computer system for well planning.

Other features and advantages of the disclosed embodiments will be or will become apparent to one of ordinary skill in the art upon examination of the following figures and detailed description. It is intended that all such additional features and advantages be included within the scope of the disclosed embodiments. Further, the illustrated figures are only exemplary and are not intended to assert or imply any limitation with regard to the environment, architecture, design, or process in which different embodiments may be implemented.

FIG. 1 is a diagram of an illustrative drilling system 100. In accordance with the present disclosure, the drilling system 100 may be used to retrieve a reservoir rock sample, such as a core sample, for characterization of a reservoir. The drilling system 100 may be one in which embodiments of the present disclosure may be implemented as part of a downhole operation performed at a well site. For example, the disclosed petrophysical modeling techniques may be performed as part of an overall seismic or other data (e.g., nuclear magnetic resonance (NMR) data) interpretation and well planning workflow for one or more downhole operations at a well site. Such downhole operations may include, but are not limited to, drilling, completion, and injection stimulation operations for recovering petroleum, oil and/or gas, deposits from a hydrocarbon bearing reservoir rock formation. As shown in FIG. 1, drilling system 100 includes a drilling platform equipped with a derrick 102 that supports a hoist 104. Drilling in accordance with some embodiments is carried out by a string of drill pipes connected together by “tool” joints so as to form a drill string 106. Hoist 104 suspends a top drive 108 that is used to rotate drill string 106 as the hoist lowers the drill string through wellhead 110. Connected to the lower end of drill string 106 is a drill bit 112 for drilling a wellbore 122 through a reservoir formation 113.

In one or more embodiments, drill string 106 may also include a reservoir rock sample collection tool (not shown) located near drill bit 112 for retrieving reservoir rock samples as the wellbore 122 is drilled through the formation. The reservoir rock sample collection tool may be designed to retrieve a reservoir rock sample 115 cut from the reservoir formation 113 by drill bit 112 as wellbore 122 is drilled through the formation. It should be appreciated that the reservoir rock sample collection tool may use any suitable mechanism to extract or collect the rock sample 115 from the formation 113. In some embodiments, the sample 115 may be cut from a side of the wellbore 122 by a separate rock extraction tool included in the reservoir rock sample collection tool and placed in a hollow storage chamber of the collection tool for later retrieval and analysis at the surface of the wellbore.

Further, in some embodiments, the collection of rock sample 115 and drilling of the wellbore 122 through rotation of the drill bit 112 may be accomplished by rotating drill string 106. The drill string 106 may be rotated by the top drive 108 or by use of a downhole “mud” motor near the drill bit 112 that independently turns the drill bit 112 or by a combination of both the top drive 108 and a downhole mud motor. During the drilling process, drilling fluid may be pumped by a mud pump 1014 through a flow line 1016, a stand pipe 1018, a goose neck 1020, top drive 108, and down through drill string 106 at high pressures and volumes to emerge through nozzles or jets in drill bit 112. The drilling fluid then travels back up the wellbore 122 via an annulus formed between the exterior of drill string 106 and the wall of wellbore 1022, through a blowout preventer (not specifically shown), and into a mud pit 1024 on the surface. On the surface, the drilling fluid is cleaned and then circulated again by mud pump 1014. The drilling fluid is used to cool drill bit 112, carry cuttings (e.g., including reservoir rock sample 115) from the borehole to the surface, and balance the hydrostatic pressure in the reservoir formation 113.

In some embodiments, the reservoir rock sample 115 retrieved from the wellbore 122 and reservoir formation 113 may be a core sample or a plug sample. As described herein, the term core sample may refer to a reservoir rock sample retrieved directly from a wellbore (e.g., wellbore 122) and/or reservoir formation (e.g., formation 113). In some embodiments a core sample may be generally cylindrical in shape and have dimensions (e.g., a diameter and a length) on the order of tens to hundreds of feet. Further, as described herein, the term plug sample may refer to a reservoir rock sample taken from a core sample (e.g., after the core sample is removed from the wellbore 122). In some embodiments, a plug sample may have a different set of dimensions from those the core sample. For instance, a plug sample may have a diameter and/or length on the order of inches or feet. While core samples and plug samples may be described herein as having particular dimensions, it should be appreciated that embodiments are not limited thereto and that a core sample or a plug sample may have any suitable dimensions.

A retrieved reservoir rock sample 115 may be used to characterize certain properties of the reservoir formation 113. In some embodiments, the retrieved reservoir rock sample 115 may be analyzed to determine a porosity of the reservoir formation 113, a presence of certain minerals within reservoir formation 113, an expected fluid flow within of the reservoir formation 113 and/or the like. In some embodiments, such analysis may be performed by physically manipulating (e.g., cutting, coring, and/or the like). Moreover, such analysis may involve the use of a core analysis tool 117, such as a permeameter, to measure or determine the properties of the sample. Additionally or alternatively, images of the reservoir rock sample 115 may be captured using an imaging device, and the resulting image data may be analyzed to determine characteristics of the reservoir formation 113. As an illustrative example, the core analysis tool 117 may be used to perform an imaging scan on the reservoir rock sample 115 to capture image data of the reservoir rock sample. In some embodiments, the image data may include a sequence of two-dimensional (2D) images of the reservoir rock sample 115 that may be combined to form a three-dimensional (3D) image of the reservoir rock sample 115. Further, the image data may include a computed tomography (CT) image, a magnetic resonance imaging (MRI) image, an ultrasound image, and/or the like. Accordingly, the core analysis tool 117 may include a suitable imaging device to capture the image data, such as a computed tomography (CT) imaging device, a microCT imaging device, an MRI imaging device, an ultrasound imaging device, and/or the like. However, it should be appreciated that embodiments are not limited thereto and that any of various imaging devices may be used as desired for a particular implementation.

While the reservoir rock sample 115 and core analysis tool 117 are illustrated proximate the drilling system 100, it should be appreciated that the reservoir rock sample 115 may be transported off location for analysis by the core analysis tool 117. In this regard, the core analysis tool 117 may be within a laboratory or at a separate geographical location away from the wellsite. Additionally or alternatively, the core analysis tool 117 may be performed in the field (e.g., proximate to the wellsite).

As further illustrated, the data from the core analysis tool 117 (e.g., the core analysis data produced by the core analysis tool 117) along with other wellsite data may be provided to a processing system 119 (e.g., a computing system). Such other wellsite data may include, for example and without limitation, production data and/or logging data captured by one or more downhole tools, e.g., a logging while drilling (LWD) tool 1026 and/or a measurement while drilling (MWD) tool 1028, coupled to drill string 106, as will be described in further detail below. The processing system 119 may use the disclosed petrophysical modeling techniques described herein to process the data and generate a model of the reservoir formation 113, which can then be used to estimate the formation's rock properties and fluid saturations. In one or more embodiments, the processing system 119 may use symbolic regression to train a machine learning (ML) model (e.g., a deep neural network) to predict petrophysical properties of the reservoir formation 113 based on the core analysis data and other wellsite data. The processing system 119 may use the trained ML model (also referred to herein as a “symbolic regression model”) to determine properties of the reservoir rock sample 115 and/or the reservoir formation 113.

In some embodiments, the processing system 119 may be implemented using any type of computing device or system, such as a computer 1040 (described further below), having at least one processor and a memory, such as a memory 121. While processing system 119 and memory 121 are shown separately from each other and separately from computer 1040 in FIG. 1, it should be appreciated that processing system 119 and memory 121 may be separate components that are integrated within computer 1040.

The memory 121 may be any suitable data storage device. Such a data storage device may include any type of recording medium coupled to an integrated circuit that controls access to the recording medium. The recording medium can be, for example and without limitation, a semiconductor memory, a hard disk, or similar type of memory or storage device. In some implementations, memory 121 may be a remote data store, e.g., a cloud-based storage location. The memory 121 may be internal or external to the processing system 119. In some embodiments, memory 121 may be used to store the core analysis data and/or wellsite data received by the processing system 119, e.g., from the core analysis tool 117 and/or the one or more downhole tools.

In accordance with the various embodiments, the one or more downhole tools may be coupled to drill string 106. In the example shown in FIG. 1, such downhole tools may include is a LWD tool 1026 and a MWD tool 1028. The distinction between LWD and MWD is sometimes blurred in the industry, but for purposes of this example, it may be assumed that LWD tool 1026 is used to measure properties of the surrounding formation (e.g., porosity, permeability), and MWD tool 1028 is used to measure properties associated with wellbore 1022 (e.g., inclination, and direction). Tools 1026 and 1028 may be coupled to a telemetry device 1030 that transmits data (e.g., well-logging data and/or a variety of sensor data) to the surface. Tools 1026 and 1028 along with telemetry device 1030 may be housed within the bottom hole assembly (BHA) attached to a distal end of drill string 106 within the reservoir formation 113. While the tools 1026 and 1028 are described as an LWD tool and a MWD tool, respectively, any suitable downhole tool may be used. To that end, as used herein, the term “downhole tool” may refer to any suitable tool or instrument used to collect information from the wellbore 122. Such a downhole tool may include any of various sensors used to measure different downhole parameters. Such parameters may include logging data related to the various characteristics of the subsurface formation (e.g., resistivity, radiation, density, porosity, etc.), characteristics (e.g., size, shape, etc.) of the wellbore 122 being drilled through the formation, and/or characteristics of the drill string 106 (e.g., direction, orientation, azimuth, etc.) disposed within the wellbore 122.

In one or more embodiments, telemetry module 1030 may employ any of various communication techniques to send the measurement data collected downhole to the surface. For example, in some cases, telemetry module 1030 may send measurements collected by the downhole tools 1026 and 1028 (or sensors thereof) to the surface using electromagnetic telemetry. In other cases, telemetry module 1030 may send the data by way of electrical or optical conductors embedded in the pipes that make up drill string 106. In yet still other cases, telemetry module 1030 may communicate the downhole measurements by generating pressure pulses that propagate via drilling fluid (e.g., mud) flowing within the drill string 106 at the speed of sound to the surface.

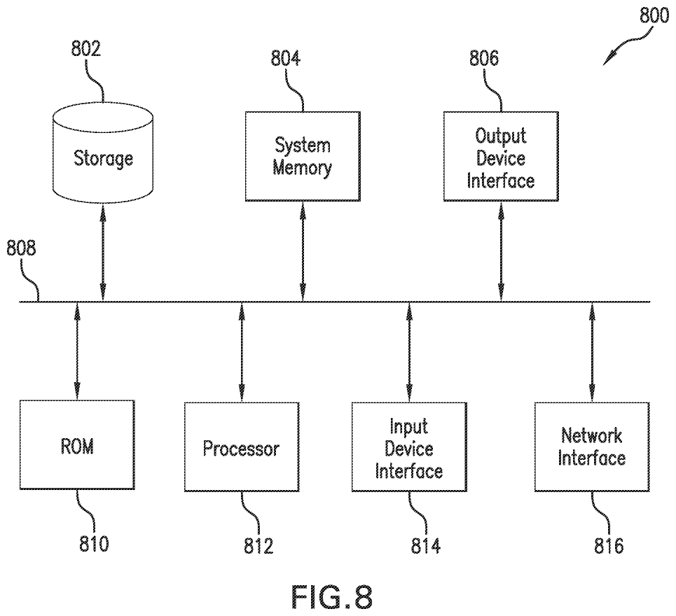

In the mud pulse telemetry example above, one or more transducers, such as transducers 1032, 1034 and/or 1036, may be used to convert the pressure signal into electrical signals for a signal digitizer 1038 (e.g., an analog to digital converter). Additional surface-based sensors (not shown) for collecting additional sensor data (e.g., measurements of drill string rotation (RPM), drilling pressure, mud pit level, etc.) may also be used as desired for a particular implementation. Digitizer 1038 supplies a digital form of the many sensor measurements (e.g., logging data) to computer 1040. Computer 1040 may be implemented using any type of computing device or system, e.g., computer system 800 of FIG. 8, as will be described in further detail below. Computer 1040 operates in accordance with software (which may be stored on a computer-readable storage medium) to process and decode the received signals, and to perform the petrophysical modeling techniques disclosed herein, e.g., for purposes of estimating reservoir rock properties (including fluid saturation) and predicting operational outcomes using drilling system 100.

In some embodiments, at least a portion of the wellsite data from the downhole tools 1026 and/or 1028 (e.g., logging data) may be forwarded by computer 1040 via a communication network to another computer system 1042, such as a backend computer system operated by an oilfield services provider, for purposes of remotely monitoring and controlling well site operations and/or performing the disclosed petrophysical modeling techniques. The communication of data between computer system 1040 and computer system 1042 may take any suitable form, such as over the Internet, by way of a local or wide area network, or as illustrated over a satellite 1044 link.

In one or more embodiments, computer 1040 may function as a control system for monitoring and controlling downhole operations at the well site. Computer 1040 may be implemented using any type of computing device having at least one processor and a memory. Computer 1040 may process and decode the digital signals received from digitizer 1038 using an appropriate decoding scheme. For example, the digital signals may be in the form of a bit stream including reserved bits that indicate the particular encoding scheme that was used to encode the data downhole. Computer 1040 can use the reserved bits to identify the corresponding decoding scheme to appropriately decode the data. The resulting decoded telemetry data may be further analyzed and processed by computer 1040 to display useful information to a well site operator. For example, a driller could employ computer 1040 to obtain and monitor one or more formation properties of interest before, over the course of, or after a drilling operation. It should be appreciated that computer 1040 may be located at the surface of the well site or at a remote location away from the well site.

To predict and/or estimate formation properties, such as saturation and rock formation factor, well logging data and/or core analysis data may be employed. These properties may be estimated using, for example, Equation 1 and Equation 2 (i.e., “Archie's Equations” or “Archie Equations”):

$$F = \frac{{R}_{0}}{{R}_{w}} = \frac{a}{{ϕ}^{m}} \qquad ( 1 )$$

$${σ}_{t} / {σ}_{w} = {ϕ}^{m} {S}_{w}^{n} / a . \qquad ( 2 )$$

In Equation 1, F is formation resistivity factor, ϕ is the total porosity, R₀ is resistivity of brine saturated rock, R_{w} is the resistivity of brine fluid, the constant a is a tortuosity factor, and the constant m is a cementation exponent. In Equation 2, σₜ and σ_{w} are the resistivity of total fluid saturated rocks, which can be mixed with hydrocarbon and water, S_{w} represents water saturation, and n represents a saturation exponent. In some embodiments, Equation 1 is applicable to 100% brine saturated rocks, and Equation 2 may be applicable to rocks saturated with both hydrocarbon and brine.

In some embodiments, development of a well logging data interpretation model begins with core analysis (e.g., data resulting from the analysis performed by core analysis tool 117). For instance, the constant a, the constant m, and/or the saturation exponent n (e.g., Archie parameters) may be determined by core analysis. Estimation of formation properties using core analysis and Archie equations (e.g., Equation 1 and Equation 2) generally works well for clean sandstones (often dubbed “Archie rocks”). For various shaly sands, source rocks containing shale oil and shale gas, or carbonates, which are considered non-Archie rocks, the parameters of Archie/Archie's equations may be fit to the saturation of these formations using core analysis with variable success. For example, the estimation of petrophysical properties, such as saturation and/or rock formation factor, may be difficult in a reservoir formation with several formation beddings with different lithology or mineralogy characteristics. In such cases, different rock facies (e.g., lithofacies and/or electrical facies) for a given layer of the formation must first be identified (e.g., from logging data, such as image logs and other logging measurements) before determining which set of Archie parameters to apply for interpreting the petrophysical properties of that particular formation layer and each of the identified facies.

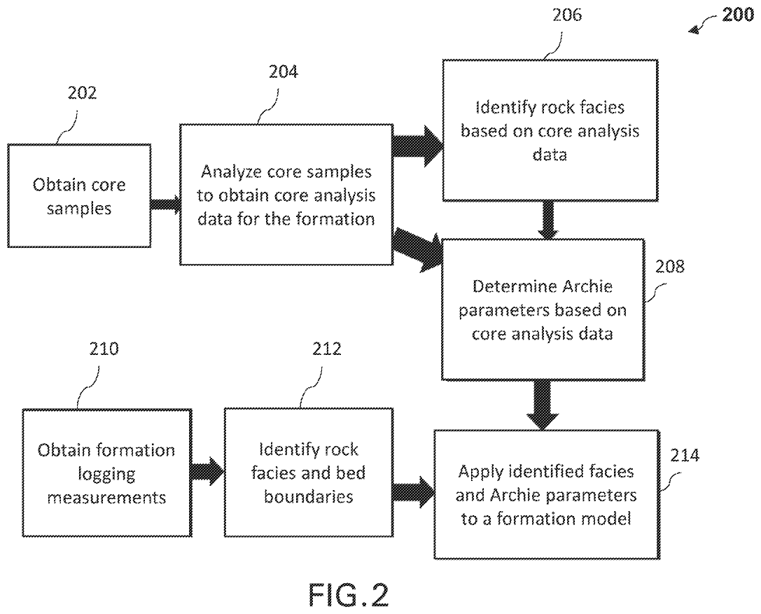

Turning now to FIG. 2, a flow diagram of a process 200 for modeling a reservoir formation and estimating formation properties using Archie's equations is illustrated. For discussion purposes, process 200 will be described with reference to drilling system 100 of FIG. 1, as described above. However, process 200 is not intended to be limited thereto.

At block 202, process 200 includes obtaining a core sample, e.g., core plugs, from a reservoir rock formation, e.g., reservoir formation 113 of FIG. 1, as described above. At block 204, the core samples are analyzed to obtain core analysis data for the formation. Such core analysis data may include, for example and without limitation, a series of petrographic thin section images along with measurements of poro-perm, resistivity index (RI), RCA, and SCA. At least a subset of this core analysis data may then be used to identify lithofacies and/or electric facies (at block 206). The remaining core analysis data may be used to determine Archie parameters (at block 208), e.g., the constants a and m along with the saturation exponent n in Equations 1 and 2, as described above, for each of the identified facies (e.g., lithofacies and/or electric facies). Facies may be identified again (at block 212) for different formation layers from well logging data, which may include image logs, and/or other logging measurements, obtained from the wellsite (at block 210). Subsequently, the facies identified at block 212 may be used to determine which of the Archie parameters from block 208 to apply for interpreting a corresponding layer of the formation (and associated facies thereof). For example, a parameter determined at block 208 for a particular lithofacie and/or electric facie identified at block 206 may be matched to a corresponding formation layer identified from the logging data at block 212. The facies identified in block 212 along with the Archie parameters determined in block 208 may then be applied to a formation model for estimating properties of the formation at block 214.

As illustrated, the process 200 may thus involve two separate identifications of facies (e.g., based on core analysis data at block 206 and logging data at block 212). The separate facie identifications may introduce error and/or inconsistencies, as facies identification may be subject to the opinion of a core analyst and/or a log analyst's opinion. Accordingly, a portion of a reservoir formation may be identified as having a first set of facies based on the core analysis, and the same portion of the formation may be identified as having a different, second set of facies based on the logging data. For instance, the boundaries of facies may differ in the second set in comparison with the first set. As such, Archie's parameters identified for the first set of facies based on core analysis data may not be suitably matched to (e.g., applicable to) the different, second set of facies. Moreover, facies variation across complex, heterogeneous formations could be continuous and mixed facies are possible. Accordingly, a discrete, limited number of facies manually defined by a user or defined without any user intervention, e.g., based on a computer-implemented training or calibration process (e.g., based on fixed parameters), may still be insufficient to model petrophysical properties of the formation. As such, the petrophysical modeling techniques of the present disclosure may be used to develop a model of the formation that can predict or estimate formation properties and saturations based on continuous logs, rather than discrete, facies-based model parameter sets.

Further, in formation evaluation practice, multiple core analysis tools (e.g., core analysis tool 117) or logging instruments (e.g., downhole tools) may be employed to derive the same petrophysical parameters, such as porosity, water saturation, clay volume, formation factor, tortuosity, cementation exponent, or saturation exponent. These different measurement tools may rely on different measurement physics. As such, derivation of the same petrophysical parameter from each of these tools may be treated independently. To that end, how two or more tool measurement inputs may be combined in a single mathematical model to derive a petrophysical parameter may be difficult and unintuitive. Moreover, conventional artificial intelligence (AI) or other data-driven modeling techniques may overfit noise or introduce bias and therefore, may not suitably honor underlying physical, lithological, or mineralogical properties of the formation and/or the tools. In addition, the lack of transparency of such “black-box” approaches make verification of the suitability of the model (e.g., in terms of honoring the above properties) and identification of the strength of correlation of particular types of measurements (e.g., from a particular tool) in different circumstances difficult. To overcome the shortcomings of such black-box approaches, the modeling techniques of the present disclosure utilize symbolic regression to develop a petrophysical model of the formation for predicting or estimating formation properties and saturations based on a variety of measurement data (e.g., collected by a variety of instruments). The disclosed techniques also account for the measurement physics (e.g., physical properties) of the various instruments (e.g., downhole tools) used to acquire the measurement data and provide a capability to evaluate and refine the model as necessary to improve the accuracy of the prediction of the formation properties and better honor the physical, lithological, or mineralogical properties of the formation and the instruments used to collect data on the formation.

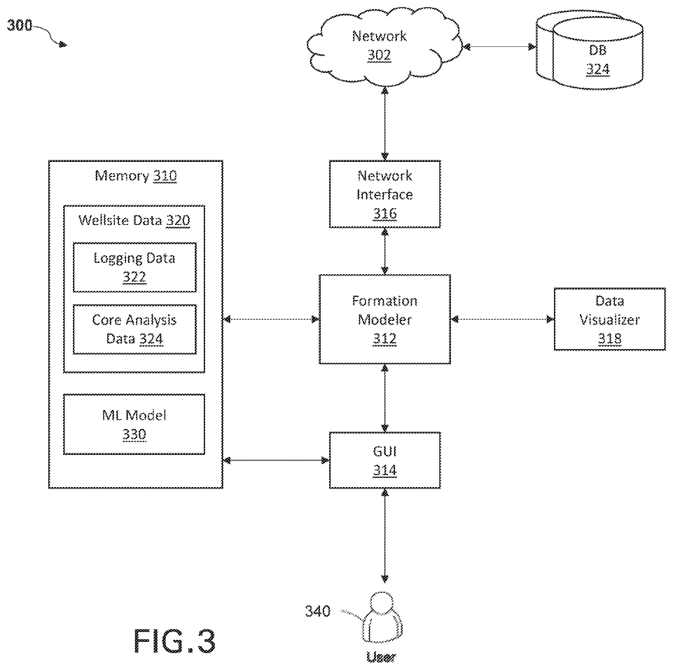

Turning now to FIG. 3, a block diagram of an exemplary system 300 for modeling a reservoir formation and its petrophysical properties using symbolic regression is illustrated. As shown in FIG. 3, system 300 includes a memory 310, a formation modeler 312, a graphical user interface (GUI) 314, a network interface 316, and a data visualizer 318. In some embodiments, memory 310, formation modeler 312, GUI 314, network interface 316, and data visualizer 318 may be communicatively coupled to one another via an internal bus of system 300. Further, in some embodiments, the components, functions, and/or operations of the system 300 may be included within and/or performed by the processing system 119 and/or the computer 1040 of FIG. 1, as described above.

System 300 may be implemented using any type of computing device having at least one processor and a memory, such as the processing system 119 and/or computer system 1040 of FIG. 1. The memory may be in the form of a processor-readable storage medium for storing data and instructions executable by the processor. Examples of such a computing device include, but are not limited to, a tablet computer, a laptop computer, a desktop computer, a workstation, a mobile phone, a personal digital assistant (PDA), a set-top box, a server, a cluster of computers in a server farm or other type of computing device. In some implementations, system 300 may be a server system located at a data center associated with the hydrocarbon producing field. The data center may be, for example, physically located on or near the field. Alternatively, the data center may be at a remote location away from the hydrocarbon producing field. The computing device may also include an input/output (I/O) interface for receiving user input or commands via a user input device (not shown). The user input device may be, for example and without limitation, a mouse, a QWERTY or T9 keyboard, a touch-screen, a graphics tablet, or a microphone. The I/O interface also may be used by each computing device to output or present information to a user via an output device (not shown). The output device may be, for example, a display coupled to or integrated with the computing device for displaying a digital representation of the information being presented to the user.

Although only memory 310, formation modeler 312, GUI 314, network interface 316, and data visualizer 318 are shown in FIG. 3, it should be appreciated that system 300 may include additional components, modules, and/or sub-components as desired for a particular implementation. It should also be appreciated that memory 310, formation modeler 312, GUI 314, network interface 316, and data visualizer 318, may be implemented in software, firmware, hardware, or any combination thereof. Furthermore, it should be appreciated that embodiments of memory 310, formation modeler 312, GUI 314, network interface 316, and data visualizer 318, or portions thereof, can be implemented to run on any type of processing device including, but not limited to, a computer, workstation, embedded system, networked device, mobile device, or other type of processor or computer system capable of carrying out the functionality described herein.

As will be described in further detail below, memory 310 can be used to store information accessible by the formation modeler 312 and/or the GUI 314 for implementing the functionality of the present disclosure. While not shown, the memory 310 can additionally or alternatively be accessed by the data visualizer 318 and/or the like. Memory 310 may be any type of recording medium coupled to an integrated circuit that controls access to the recording medium. The recording medium can be, for example and without limitation, a semiconductor memory, a hard disk, or similar type of memory or storage device. In some implementations, memory 310 may be a remote data store, e.g., a cloud-based storage location, communicatively coupled to system 300 over a network 302 via network interface 316 (e.g., a port, a socket, an interface controller, and/or the like). Network 302 can be any type of network or combination of networks used to communicate information between different computing devices. Network 302 can include, but is not limited to, a wired (e.g., Ethernet) or a wireless (e.g., Wi-Fi or mobile telecommunications) network. In addition, network 302 can include, but is not limited to, a local area network, medium area network, and/or wide area network such as the Internet.

In some embodiments, memory 310 may be used to store wellsite data 320. Wellsite data 320 may include, for example, logging data 322 (e.g., image logs and/or other logging measurements) and core analysis data 324 associated with a reservoir formation, e.g., formation 113 of FIG. 1, as described above. It should be appreciated, however, that embodiments are not limited thereto and that memory 310 may be used to store other types of data (e.g., production data) associated with the reservoir formation (or one or more wellsites thereof). Such data may have been collected by a variety of different tools. Accordingly, logging data 322 in memory 310 may include data collected by any number of downhole logging tools, and core analysis data 324 may have been collected by any number of core analysis tools. The different tools, e.g., core analyzers and well logging instruments, used to collect this data may be characterized by different measurement physics, which may cause the measurement values obtained for the same set of formation properties to vary depending on the tool that is used. Logging data 322 may include, for example, well logging measurements, e.g., as collected by LWD tool 1026 and MWD tool 1028 of FIG. 1, as described above. Core analysis data 324 may include, for example, NMR, resistivity, induction, acoustic, density, photoelectric (PE) data, spontaneous potential (SP) data, natural gamma ray, neutron, logs, and/or the like, e.g., as obtained from the analysis of a core sample by core analysis tool 117 of FIG. 1, as described above. In some embodiments, the system 300 may be communicatively coupled to a downhole tool and/or a core analysis tool via network 302. Accordingly, logging data 322 and core analysis tool 324 may be obtained from the downhole tool and the core analysis tool, respectively, over network 302 via network interface 316 of system 300. In some embodiments, the wellsite data 320 may be obtained from a remote database 324, which may be accessed over network 302 via the network interface 316.

In one or more embodiments, the formation modeler 312 may utilize symbolic regression for training a machine learning (ML) model (e.g., a deep neural network) 330 to determine a model for estimating properties of the reservoir formation, based on wellsite data 320. The model determined by the trained ML model (or symbolic regression model) 330 may be formulated as, for example, a mathematical expression, equation, or function representing the formation's properties. Examples of such formation properties include, but are not limited to, Archie's parameters, saturation, formation resistivity factor, and/or the like. Thus, wellsite data 320, including logging data 322 and core analysis tool 324, in this example may serve as training data for modeling the reservoir formation, i.e., by training ML model 330 to determine an appropriate formation model. As shown in FIG. 3, ML model 330 may also be stored in memory 310. In one or more embodiments, the symbolic regression used by formation modeler 312 may be in the form of a model selection algorithm that is capable of improving a population of candidate models. In one or more embodiments, the underlying algorithm of the symbolic regression may mimic genetic evolution processes that consist of iteratively performing crossover and mutation operations. Crossover may involve randomly merging or combining two candidate models into two new candidate models. Mutation may involve making a random change to at least a part of an individual candidate model to create a new candidate model and associated function. The associated function may be, for example, a set of equations or mathematical expressions corresponding to a child population of candidate models. The functions/equations defining the child candidate models may be derived by randomly perturbing or varying one or more parameters of the corresponding functions/equations used to define the models in a parent population. Such parameters may include, for example, one or more coefficients, constants, exponents, etc. of the corresponding function/equation. In some implementations, the parameters may correspond to different Archie parameters of an Archie's Equation, e.g., Equation 1 or Equation 2, as described above. Iterative mutations and crossovers in the symbolic regression may eventually produce an optimized target function (e.g., a mathematical expression) that defines a corresponding model of the reservoir formation.

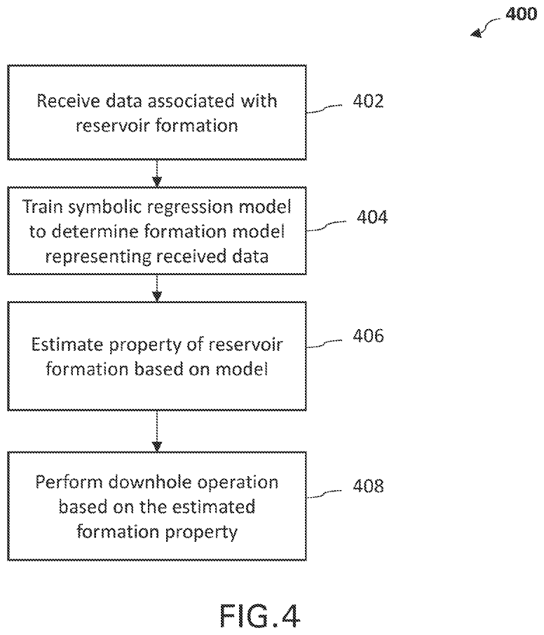

In some embodiments, the ML model utilized by formation modeler 312 may be trained using a variety of different types of data associated with the reservoir formation, such as logging data 322 and core analysis data 324, e.g., from a variety of different data sources (e.g., different logging and/or core analysis tools) to estimate a property of the formation that is not included in the data. For instance, the ML model 330 may be trained to use data collected from various instruments, such as a core analysis tool and a downhole tool (e.g., a logging tool), to identify a candidate model that corresponds to the data from each of the instruments. To that end, the formation modeler 312 may combine measurement data corresponding to different physics to determine a model (e.g., as defined by a mathematical expression) that enables a petrophysics practitioner to evaluate, justify, and validate whether the model honors the various physics of the formation. In that regard, the crossover of input data coming from two different measurement physics, e.g., as performed by the formation modeler 312 using symbolic regression, may integrate different data sets, and the mutation process performed by the formation modeler 312 may produce an optimized model resulting from this integration. Further details regarding the use of symbolic regression for training an ML model, such as ML model 330, (also referred to herein as a “symbolic regression model”) to determine an optimal formation model (e.g., as selected from a population of candidate models) and estimate formation properties using such a model will be described in further detail below with respect to FIGS. 4-7.

In some embodiments, the property of the formation may be represented by an Archie parameter, such a tortuosity coefficient, a cementation exponent, or a saturation exponent associated with the reservoir formation, as described above. Additionally or alternatively, the property may be a porosity, permeability, capillary pressure, bound fluid volume, shale volume, productivity index, relative permeability, effective permeability, hydrocarbon properties, formation salinity, and/or the like. Further, in some cases, the system may further manipulate or use an estimated property to determine a further property.

In some embodiments, the system 300 may output the estimated property of the reservoir formation. In some embodiments, the property of the reservoir formation may be provided as a numerical indication, a graphical indication, a textual indication, or a combination thereof. For instance, the property of the reservoir formation may output to and/or by the GUI 314, and/or the data visualizer 318. For instance, the property of the reservoir formation may be output to the GUI 314, which may be provided on a display (e.g., an electronic display). The display may be, for example and without limitation, a cathode ray tube (CRT) monitor, a liquid crystal display (LCD), or a touch-screen display, e.g., in the form of a capacitive touch-screen light emitting diode (LED) display. Further, the data visualizer 318 may be used to generate different data visualizations, such as bar graphs, pie graphs, histograms, plots, charts, numerical indications, textual indications, and/or the like based on the property of the reservoir formation. The data visualizer 318 may further perform any suitable data analysis on the property of the reservoir formation, such as interpolation, extrapolation, averaging, determining a standard deviation, summing or subtracting, multiplying or dividing, and/or the like. Moreover, the data visualizer 318 may be used to visualize a model of the reservoir formation based on the estimated property of the reservoir formation and/or the wellsite data 320 (e.g., logging data 322 and/or core analysis data 324). In some instance, the formation model may be visualized as a 2D or a 3D model within GUI 314.

In some embodiments, GUI 314 enables a user 340 to view and/or interact directly with the modeled reservoir formation or properties thereof. For example, the user 340 may use a user input device (e.g., a mouse, keyboard, microphone, touch-screen, a joy-stick, and/or the like) to interact with the modeled parameters of the reservoir formation via the GUI 314. In some embodiments, the GUI 314 may receive a user input via such a device to modify, accept, or reject the estimated property of the reservoir formation. Moreover, in some embodiments, such a user input may alter the training and/or output of the formation modeler 312, as described in greater detail below. The GUI 314 may additionally or alternatively receive a user input to generate the model, to generate a particular data visualization (e.g., via the data visualizer 318), to run a particular simulation with the model, to adjust a characteristic of the model and/or a data visualization, and/or the like.

While certain components of the system 300 are illustrated as being in communication with one another, embodiments are not limited thereto. To that end, any combination of the components (including memory 310, formation modeler 312, GUI 314, network interface 316, and data visualizer 318) illustrated in FIG. 3 may be communicatively coupled via an internal bus of system 300.

FIG. 4 is a flowchart of an illustrative process 400 for determining a property of a reservoir formation using a symbolic regression model. For discussion purposes, process 400 will be described with reference to FIG. 1 and the system 300 of FIG. 3. However, process 400 is not intended to be limited thereto.

In block 402, the process 400 receiving wellsite data (e.g., wellsite data 320 of FIG. 3, as described above) associated with the reservoir formation. The data may be received by the system 300, for example. Moreover, the data may be collected by one or more downhole tools, such as tools 1026 and 1028, as illustrated in FIG. 1, which may each be associated with respective physics and/or physical properties. For instance, the data may include core analysis data, such as core analysis data 324, and/or logging data, such as logging data 322 of FIG. 3, as described above. To that end, logging data obtained by a downhole tool positioned within a wellbore associated with the reservoir formation may be received, and/or core sample data associated with a core sample and obtained by a core analysis tool different than the downhole tool may be received. Further, the logging data or the core sample data may include, but is not limited to, NMR data, resistivity data, induction data, acoustic, density data, photoelectric (PE) factor data, spontaneous potential (SP) data, natural gamma ray data, neutron data, or logs.

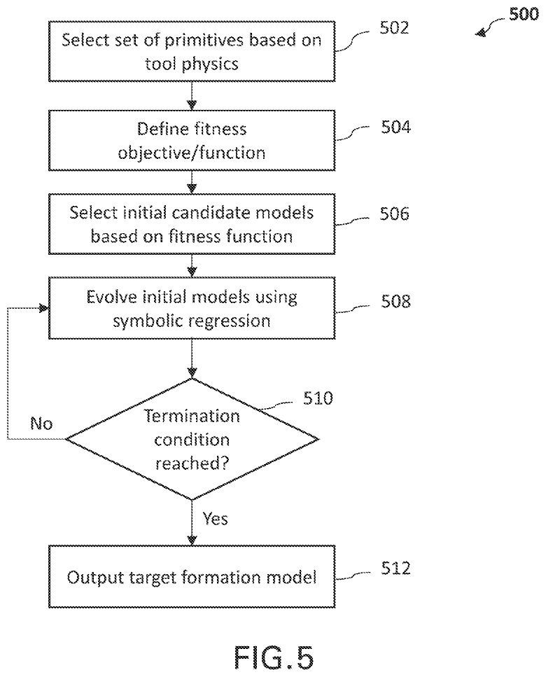

In block 404, the process 400 involves training a symbolic regression model (e.g., a machine learning model using symbolic regression), such as ML model 330 of FIG. 3, as described above, to determine a formation model representing the data received in block 402. In one or more embodiments, the symbolic regression model may be trained to generate a plurality of different candidate models based on the received data (e.g., logging data and/or core sample data). Additional details of training the symbolic regression model are provided in FIG. 5.

With reference now to FIG. 5, a flowchart of an illustrative process 500 for training a symbolic regression model, e.g., in accordance with block 404 of FIG. 4, is shown. For discussion purposes, process 500 will be described with reference to FIG. 1, the system 300 of FIG. 3, and FIGS. 4, 6, and 7. However, process 500 is not intended to be limited thereto.

In block 502, the process 500 may involve selecting a set of primitives based on tool physics. The primitives may be referred to as initial genotypes and may include functions, operators, parameters, and/or variables. In particular, the primitives set may include functions, operators, parameters, and/or variables associated with one or more properties of the tools used to collect data used to train a machine learning model (e.g., ML model 330 of FIG. 3) using symbolic regression. When data from more than one measurement tool (e.g., data from downhole tool 1026 or 1028 and core analysis tool 117 of FIG. 1, as described above) is involved, the selected primitives set may include elements that define the relationship of these measurements. The primitives set may therefore represent measurement characteristics of the tools that affect the formation data acquired by each tool and thus, serve as a building block for the evolution of the function (e.g., model) output by the formation modeler 312 based on such data. The elements in the primitives set can be represented as a tree, a list, or another mathematically transparent form. In the illustrated embodiment, the tool measurement physics is used in the primitives set, which may limit an initial construction of function search space for the symbolic regression model.

In some embodiments, selecting the primitives set may involve automatically determining, based on the logging data or identification of the downhole tool used to collect this data, a first primitive set. The selecting of the primitives set may further involve automatically determining, based on the core sample data or identification of the core analysis tool used to collect this data, a different, second primitive set, and the first primitive set and the second primitive set may be applied to the symbolic regression model.

The primitives in the selected set may also be used as constraints in crossover and mutation (e.g., at block 508, as described in further detail below). For instance, tool measurement physics can be used to define those features that are preferred to be retained instead of removed or replaced during crossover and/or mutation. In that regard, defining relationships or features that are relatively strong, such as NMR T2 being proportional to pore size, permeability being inversely proportional to bulk volume irreducible, and/or the like to be retained may result in physics of the tool measurements being obeyed and a computational efficiency of the model 312 being improved over cases where these features are not retained.

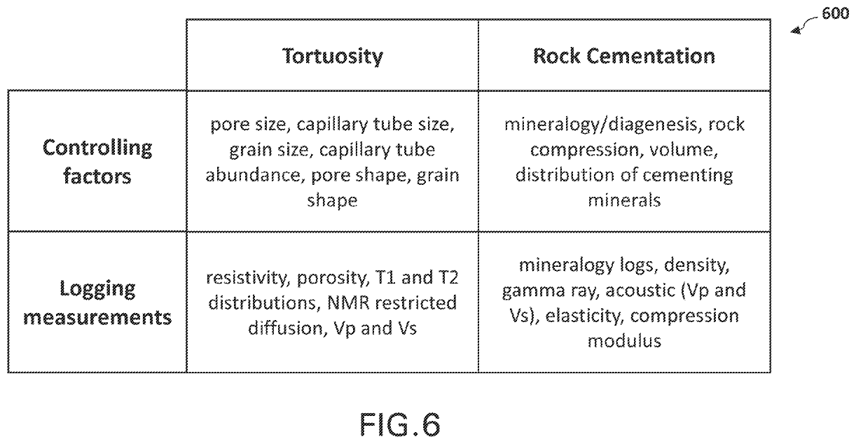

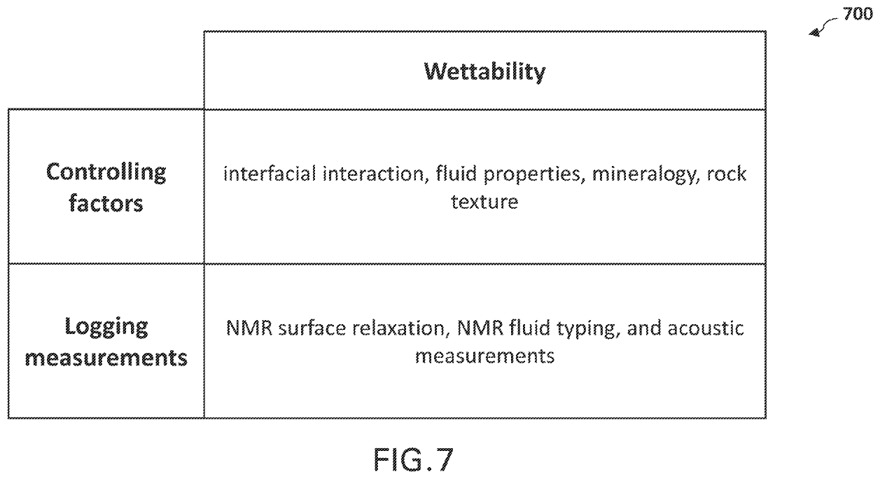

In some embodiments, the data and/or primitives set(s) used to train the symbolic regression model may depend on the data available and/or the property being predicted by the symbolic regression model and/or by the model output produced by the symbolic regression model, as described in greater detail with reference to FIGS. 6 and 7. FIG. 6 is a table 600, which provides an illustrative example of a dataset useful for training the symbolic regression model, e.g., for selecting the primitives in a set and defining relationships between the selected primitives.

In some embodiments, the symbolic regression model may be used to predict the cementation exponent m, the saturation exponent n, or another of Archie's parameters from logging data. The training data for the symbolic regression model may include core analysis data alone, logging data alone, or a mixed core analysis and logging data. In the case of training data including core analysis data alone, m, n, and the Archie model constant can be directly determined from appropriate resistivity or conductivity measurement of brine, core plugs fully saturated with brine, and core plugs saturated with different fraction of hydrocarbon, So, and the brine, Sw=1−So. The values may be determined according to Equations (1) and (2), for example and may be considered a benchmark or “ground truth” target in the training process.

Further, to develop a formation model, e.g., a petrophysical model, which is applicable to using logging measurements as inputs, additional core analysis data measurement inputs that are equivalent to logging measurements may be used. In consideration of which logging data may be more relevant to the cementation factor m and the tortuosity constant a, logging measurements that may respond to tortuous flow pathways and rock cement may be evaluated. As shown in FIG. 6, the main controlling factors of tortuosity are the pore size, capillary tube size, grain size, and capillary tube abundance, as well as pore shape and grain shape. The logging measurements that correlate to tortuosity include resistivity, porosity, T1 and T2 distributions, NMR restricted diffusion measurement, and seismic velocity parameters (Vp and Vs), among others. As further shown in FIG. 6, the main controlling factors of rock cementation include mineralogy/diagenesis, rock compression, volume and distribution of cementing minerals. The logging measurements that may correlate to these attributes include mineralogy logs, density, gamma ray, acoustic (Vp and Vs), and elasticity and compression modulus.

The dataset illustrated in FIG. 6 may be used at the start of the formation model development process using the symbolic regression model. In some embodiments, not all of such logging data is available, and so a subset of data may be used. In general, tortuosity is primarily a direct measurement of pore space and pore fluid, thus a measurement that is primarily sensitive to pore and pore fluid, such as NMR, may be preferred. Cementation is primarily a rock matrix measurement, therefore a type of logging measurements that is dominantly reflective of the matrix properties, such as acoustics or nucleus may be desired. Since the tortuosity and cementation are related, a combination of at least one primary pore response and one primary matrix response may be employed with the symbolic regression model. In that regard, the primary response, NMR: pore fluids, of the tortuosity and/or the measurements associated with the tortuosity may be combined with the primary response, acoustics/nucleus: rock matrix, of the cementation of rock and/or the measurements associated with the cementation of the rock for the symbolic regression model.

Turning now to FIG. 7, a table 700 illustrates controlling factors of wettability and related logging measurements are shown. The saturation exponent n involves multiphase fluids in pore space of a rock. The saturation exponent describes the dependency on the presence of non-conductive fluid, such as hydrocarbon fluid, in the pore space in different wettability characteristics of the rock. In a water-wet rock, a thin film of water on the surface of the pores that are occupied by oil, thus the brine phase forms a continuous phase thus has higher conductivity. In an oil-wet rock, water is the discontinuous phase in the form of droplets within the pore space, which lowers the conductivity. Wettability is largely controlled by minerology and mineral distributions in rocks, the viscosity and composition of the fluid saturating the rock, the texture of pore surface especially surface roughness, and interfacial interactions between pore fluid to grain. For the purpose of predicting n, it may not be required to explicitly determine wettability index from logs. For instance, in addition or the alternative to determining the wettability index, the types of logging data affected by wettability changes are identified and used to predict n.

The microscopic interfacial interaction may affect the NMR surface relaxivity, which may affect NMR relaxation time distribution response. Conductivity measurement is affected by wettability but may benefit from separation from other conductivity effects, such as clay content, type, and distribution, as well as the salinity of the formation fluid and in microlateral logging, the near wellbore invaded mud filtrate fluid salinity. Because n may eventually be used for interpreting EM logs for saturation determination, these logs may not explicitly be used as primary inputs for predicting the saturation exponent n.

The fluid properties, particularly oil viscosity and composition (such as SARA analysis) can be directly obtained from fluid sampling but usually they are only obtained at limited, discrete depths. On the other hand, NMR 1D, 2D, and/or 3D fluid typing can provide a large bulk of fluid properties.

Mineralogy and mineral distribution can be obtained by nucleus logging, including, but not limited to, density, gamma ray or spectral gamma ray, element spectroscopy, and other logging data. NMR and acoustic logs can be affected by rock texture.

Logging data sensitive to these four control factors (e.g., interfacial interaction, fluid properties, mineralogy, and texture) may preferably be selected for use by the symbolic regression model. In some embodiments, data sensitive to a subset of these control factors may alternatively be used to predict n using the symbolic regression model. Many of these logging measurements have equivalent laboratory core analysis measurements which, together with the laboratory RI (resistivity index) measurements and saturation measurements derived “ground-truth” n (e.g., based on core analysis), may be used to train the symbolic regression model to develop an appropriate formation model.

Returning now to FIG. 5, in block 504, the process 500 may involve defining a fitness objective. The fitness objective (or fitness function) may be a function used to evaluate a product of the formation model being developed. The fitness objective may consist of a minimization process (e.g., mean-squares error minimization or root mean-squares error minimization) but can be other forms. In some embodiments, the fitness objective may include additional components (e.g., a penalty function) to reduce formation model complexity and prevent overfitting the data. In some embodiments, an overcomplicated formation model may fit to noise instead of main formation and fluid features. Further, well logging data acquired in downhole operation often may have noise contamination significantly higher than laboratory core analysis measurement, and different logging measurements may have different degrees of noise contamination. Accordingly, the physics of tool measurements can be used to design the penalty function. Optionally, the fitness objective can include a regularization component to stabilize the predicted solution or target function output by the symbolic regression model and used to define a petrophysics model of the formation. In some embodiments, the target function may be based on the coefficients or parameters associated with a known or predefined base function, such as an Archie equation, where the general form of the function may be preserved but coefficients (e.g., constants, exponents, etc.) in the equation may vary, e.g., by applying symbolic regression over multiple iterations to yield prediction (or candidate) equations to capture the variations of these coefficients. Such coefficient variations may be representative of logging responses collected from the underlying formation being modeled. Alternatively, the target function may be derived directly from the training data (e.g., logging data inputs) applied to the symbolic regression model without using a predefined base function. The target function in this case may be, for example, a new petrophysical equation that does not resemble the form of any known equation for a given petrophysical parameter.

In block 506, the process 500 may involve selecting one or more initial candidate models for the formation or one or more functions defining an initial parent population of candidate formation models, based on the fitness objective/function. In one or more embodiments, a term may be added or removed from a candidate function selected by the symbolic regression model based on the fitness objective (e.g., based on a fitness function score assigned to each term of the function) to improve the formation model being developed.

In block 508, the process 500 may involve evolving the initial population of models using symbolic regression. In one or more embodiments, block 508 may include performing evolutionary computation using the symbolic regression model discussed above to find a formation model that best fits the training data, e.g., the selected primitives from block 502 in combination with wellsite data (e.g., logging data 322 and core analysis data 324 of FIG. 3). The main algorithm in the symbolic regression model may be an evolutionary algorithm or program that iteratively performs crossover and mutation operations to generate new genotypes (e.g., intermediate functions and corresponding candidate petrophysical models of the formation) at each iteration until a predetermined termination condition is satisfied or reached (at block 510). The genotypes may correspond to a child population of candidate models derived from the initial or preceding iteration's parent population, as described above. In some embodiments, a model selection process may be conducted to select a subset of candidate models from among the intermediate population of models generated at each iteration before crossover and mutation process is applied for the next iteration. The purpose of such a selection process may be to ensure the best formation models are selected as parents for each intermediate population that is generated while the crossover and mutation processes are used to explore the candidate model space. Crossover retains strong features, and mutation process explores model space with respect to the intermediate petrophysical model formula until a predefined termination condition is reached (e.g., at block 510). In some embodiments, constraints may be imposed during the crossover and mutation process such that a new intermediate model (e.g., a petrophysical model equation) after crossover and mutation has the same units on either side of the equation.

In block 510, the process 500 may involve determining whether a termination condition has been reached. The termination condition may be any predefined condition. The predefined termination condition may include, for example, a predefined fitness score (e.g., associated with the fitness objective). Additionally or alternatively, the termination condition may be a threshold number of generations (or iterations of block 508) that elapse without further or enough improvement (e.g., based on the fitness objective) of the petrophysical model generated by the symbolic regression model. Exploring model space during the crossover and mutation process can become excessively tedious depending on the complexity of the model. Thus, in order to improve computational efficiency, the fitness function (or fitness objective) may be dynamically modified to add a penalty for model complexity, as described above. Further, as described above, by retaining strong features in the crossover process, a more efficient process (e.g., improved computational efficiency) for building the targeted interpretation model may be provided, and the targeted model may better obey physics.

If, at block 510, the termination condition has not been reached, the process 500 may return to block 508. If, on the other hand, the termination condition has been reached, the process 500 may proceed to block 512. At block 512, the process 500 may involve outputting a target formation model (e.g., a petrophysical model) that corresponds to (or best fits) the tool data. The target model may be defined by a target function in the form of a mathematical expression, for example. As such, the output model may be evaluated and/or validated against properties of the formation and/or the tools.

Once the termination condition is reached (e.g., at block 510), at least one mathematical expression representing may have been obtained by the symbolic regression model. Accordingly, at block 512, the process 500 may involve outputting the mathematical expression (e.g., a petrophysical model) that was generated by the symbolic regression model. In some cases, more than one model is generated by the regression model that meets the termination condition. To that end, the symbolic regression model may output a single model or multiple models. For instance, based on a ranking of the models meeting the termination condition, the symbolic regression model may output (e.g., select) a model with a best-fit. In cases where multiple models are output (e.g., at block 512), the expressions may be evaluated and/or examined to select a particular model, which may not be possible with conventional data-driven modeling approaches, as described above. That is, for example, a user may provide a user input to select a candidate model from among one or more models. In some embodiments, a particular model that does not overfit or violate the underlying measurement physics may be selected for estimating properties of the reservoir. Moreover, since the symbolic regression model may generate mathematical expressions over iterative evolutions (e.g., block 508), candidate models may be examined in an intermediate form (e.g., before termination condition 510 is satisfied) within the whole population of models, which may be helpful to determine the reasonableness of a candidate model in terms of obeying measurement physics. To that end, while evaluation of the intermediate models is not necessarily required, it may provide a means of ruling out models with an overfitting issue, particularly due to noise contamination or systematic measurement errors.

If the candidate model output at block 512 is satisfactory, the model development process 500 is stopped. A user may accept the candidate model as satisfactory by providing a user input via the GUI 314, for example. To that end, the candidate model may be selected from among a group of candidate models. Additionally or alternatively, the candidate model may be identified as satisfactory based on the model obeying certain physics, correlating to data (e.g., test or validation data) within a certain threshold, and/or the like. A candidate model may be less than satisfactory for a variety of reasons. For instance, for multiphysics measurements, correlation of the candidate models with one or more of the measurements may be weak (e.g., below the threshold). In some embodiments, the candidate model may show an opposite or unreasonable dependency between terms and/or parameters. Further, in some embodiments, the candidate model may be too sensitive to a variable change and may thus be too sensitive to is measurement uncertainty.

The correlation of the candidate models with one or more of the measurements being weak may indicate that the weakly correlated measurements should be removed from the model to simplify the model. As an illustrative example, in logging interpretation, one less well logging tool may be used to obtain the same petrophysical information. This simplification may not be readily apparent using other blackbox machine learning approaches that do not provide a model. In this way, the symbolic regression model advantageously may provide understanding and adaptability of the output model, which is not available via other modeling approaches.

The correlation of the candidate models with one or more of the measurements being weak may additionally or alternatively indicate that the training dataset is not large enough. That is, for example, the available training data may only represent a narrow envelope of formation characteristics, which may not adequately correspond to other formation characteristics. In such cases, adding more and different types of formations for retraining the symbolic regression model may increase the application envelope of the output model and the resulting correlation of the model.

The candidate model may show an opposite or unreasonable dependency if the initial primitives set is restrictive or if the training data quality is lacking. A corrective action for a restrictive primitives set may be to increase the search space of the symbolic regression model, and a corrective action for issues with training data quality may be to leave different portions of the training data out to determine which or if any of the portions are unsuitable for training the symbolic regression model.

Over-sensitivity often occurs for a part of data space instead of over the entire data space. To that end, the candidate model may too sensitive to a variable change may due to this uneven distribution of over-sensitivity. As an illustrative example, if one petrophysical parameter is found to be inversely proportional to porosity, then the model could be very sensitive to extremely low-porosity formation. In this case, the application envelope may be improved if the near-zero porosity is excluded. Another example is a petrophysical parameter that shows a power-law dependent to a measurement quantity. In this case, the model may become overly sensitive as exponent increases. Continuing with this example, setting an upper limit of the exponent to a physically reasonable value may limit the over-sensitivity of the model. In some embodiments, changing a functional form may reduce the oversensitivity of the model significantly. Accordingly, in some embodiments, a candidate model that avoids oversensitivity may be provided by re-defining the primitives set and retraining the data. For instance, the 1/porosity function may be removed from the primitives set and may be replaced with 1/(porosity+ε), where ε is a small constant that may not make much difference when porosity is large but may limit the value to 1/ε for zero porosity formation. Re-defining or changing the primitives set in cases where the model shows weak correlation or shows unreasonable dependencies may also be useful to attempt to generate an improved model or to determine differences between models generated based on different primitives sets.

The above examples of less than satisfactory candidate models and courses of action to correct the candidate model are intended to be exemplary and not limiting. In that regard, a candidate model may be optimized with the symbolic regression model using any suitable approach.

Returning now to FIG. 4, at block 406, the process 400 may involve estimating at least one property of the reservoir formation based on the model associated with the data. In particular, the property may be determined based on the model produced by the symbolic regression model (e.g., the mathematical expression output at block 512) using data from one or more tools. In some embodiments, the data received at block 402 may lack the property of the reservoir formation. That is, for example, the property may not be determined directly from the data in some embodiments. Examples of formation properties that may be estimated using the model include, but are not limited to, a porosity, permeability, capillary pressure, bound fluid volume, shale volume, saturation, productivity index, relative permeability, effective permeability, hydrocarbon properties, formation salinity. In some cases, the property may be an Archie's parameter, such as tortuosity factor, cementation exponent, or a saturation exponent. The property may be a saturation of reservoir rock associated with the subsurface reservoir formation. Further, the property may be an electrical efficiency parameter of reservoir rock associated with the subsurface reservoir formation, as described in greater detail below.

As described above, the symbolic regression model may output a model (e.g., a petrophysical model expressed as a mathematical equation or equations). This model may be used to determine a property of a reservoir directly in some embodiments. For instance, using the model and data (e.g., logging data and/or core analysis data), a property of the reservoir, such as those listed above, may be determined. As an illustrative example, saturation may be directly determined using the model and the data. In some embodiments, a further derivation may be utilized to determine the property. Continuing with the example of saturation, the model may be used to determine the cementation constant, tortuosity constant, or saturation constant (e.g., Archie's parameters), which may then be used to determine the saturation. To that end, the value of predicting Archie's parameters is that they may provide an indicator of how far rock characteristics depart from ideal “Archie rock,” in addition to their ability to be employed in a determination of saturation. However, for saturation determination, it is not necessary to use the Archie equation's as a bridge between logging measurements and saturation determination. The same set of logging data, plus electromagnetic logs (resistivity or induction), can serve the purpose using the techniques described herein.

Further, modeling has been described herein with respect to Equations 1 and 2, as described above, and/or parameters contained therein, the techniques described herein may additionally or alternatively be applied to other equations. To that end, use of Archie's equations (e.g., Equations 1 and 2) is one method to interpret conductivity data for wetting phase fluid saturation estimation in rocks, but this is not the only way of obtaining saturation from conductivity. Further, the Equations 1 and 2 may have limited accuracy even with calibrated C, m, and n for formation rocks that are significantly non-Archie type, such as shaly sands, because the conductivity of shale cannot be very well compensated by adjusting m, n, and a. Moreover, mathematically, high m and n values can make the equations overly sensitive to measurement uncertainties, as m and n are exponents (e.g., are associated with power law). For instance, for certain formations, especially for some carbonate formations, the cementation exponent can be as high as 5. For strongly oil-wet formation, the saturation exponent can be as high as 8. In such cases, it may be desirable to use different parameters to describe these situations.

Another approach for modeling reservoir formation properties is an electric efficiency model, as shown in Equations 3 and 4 below.

$$\frac{{σ}_{t}}{{σ}_{w}} = ( Sw \cdot ϕ ) ( {e}_{t} {E}_{0} ) \qquad ( 3 )$$

$$\frac{{σ}_{0}}{{σ}_{w}} ( ϕ {E}_{0} ) + {c}_{0} \qquad ( 4 )$$

In Equations 3 and 4, the term E₀ represents a brine geometric distribution factor, the term eₜ represents an HC emplacement modification of the brine geometric factor, the term c₀ represents a departure from Archie's rocks (residual conductivity at zero porosity), and the remaining terms correspond to their equivalents described in Equations 1 and 2. With the electric efficiency model, the conductivity ratio can be expressed as the product of volumetric fraction of conductive rock (Sw·ϕ), and some quantities related to electrical efficiency parameters may be expressed as (eₜE₀), where the efficiency is defined as the conductivity ratio between the standard tube model and the actual rock. In the electrical efficiency equations (e.g., Equations 3 and 4), the conductivity of shale can be conveniently described as one additional term c₀ representing normalized conductivity in a zero-porosity rock. The term c₀ thus represents conductivity from a rock matrix rather than fluid. Thus, the conductivity ratio of a brine saturated rock can be considered as the summation of one term representing contribution from fluid (ϕE₀) and one term representing contribution from matrix, c₀. Further, the term E₀ is only related to single brine phase saturated rock, and the term eₜ may only affect multiphase saturated rock. These two fluid geometric factors are related to the tortuosity of the rocks, and eₜ is further affected by wettability. Thus, the training data described in FIGS. 6 and 7 may also be suitable as the training data for the electrical efficiency model prediction using the symbolic regression model. That is, for example, the techniques described herein may be used to determine any of the parameters included in Equations 3 and 4.

Equations 1 and 2 include two exponent parameters (e.g., m and n), while Equations 3 and 4 include two multiplier parameters (e.g., the electrical efficiency parameters). Accordingly, the sensitivity (and over sensitivity) of Equations 1 and 2 is different than that of Equations 3 and 4 in the formation space. Since the electric efficiency model and Archie's model have different sensitive domains, and they may handle non-Archie rock types (e.g., shaly sands) differently, these two models may be effectively combined together and optimized using the symbolic regression model (e.g., using the techniques described herein). To that end, in some embodiments, the Archie model and the electrical efficiency model (e.g., Equations 1-4) may be used as the primitives set at block 502, and the symbolic regression model may be applied upon the primitives set to obtain the final solution equations (e.g., output at block 512). In this regard, crossover and mutation of these models may occur at block 508, and the resulting function may then be optimized (e.g., over iterations of block 508) and output at block 512. The final solution may be more similar to the form of the Archie model, more similar to the form of the electric efficiency model, a function combined from weighting both models with a significant weight, or an equation form completely different from the two models.

In some embodiments, the same input data may be used to predict m, n, E₀ and eₜ simultaneously. In this way, the petrophysical model output by the symbolic regression model may confirm both the Archie model and the electrical efficiency model. In some embodiments, a weighting scheme may be added to m, n, E₀ and/or eₜ depending on which one model is more accurate at for specific characteristics of a formation.

Further, in some embodiments, the model output by the symbolic regression model may be suitable to determine the property of the reservoir formation over a continuous set of values. In this regard, the model may be used to determine the property over a heterogeneous subsurface reservoir formation where the value of the property varies over different portions of the formation. For instance, the values may be determined using continuous log data. Moreover, boundaries of the different portions, which may correspond to different facies, may be identified based on variation of the value of property over the formation. In this way, any number of facies and/or thresholds between property values defining different facies may be employed to characterize a formation.

At block 408, the process 400 may involve performing a downhole operation, based on the property of the formation estimated in block 406. Examples of such a downhole operation include, but are not limited to, a drilling operation to drill a wellbore or portions thereof along a planned path through the formation, a completion operation along one or more sections of such a wellbore path, and a stimulation operation involving fluid injection to stimulate hydrocarbon production from the formation surrounding one or more sections of a wellbore drilled along its planned path. In some implementations, the downhole operation may be a drilling operation in which the estimated formation property may be used to adjust the planned path of the wellbore being drilled. For example, the planned path may be adjusted by adjusting one or more operating parameters of a drill string (e.g., drill string 106 of FIG. 1, as described above) disposed within a wellbore being drilled along its planned path through the formation. The parameters may be adjusted, for example, by transmitting control signals representing the appropriate adjustments from a surface computing device (e.g., computer 1040 in FIG. 1) to a downhole controller coupled to a bottom hole assembly of the drill string. The signals received by the downhole controller may then be used to adjust the trajectory of the drill string and the path of the wellbore as it is drilled through the formation. It should be appreciated that any of various communication techniques, e.g., telemetry, may be used to transmit the control signals downhole and that the drill string may include any of various components (e.g., telemetry device 1030 of FIG. 1) for enabling the functionality described herein.

In one or more embodiments, process 400 may also include outputting the estimated property of the model for a user (e.g., user 340 of FIG. 3) at a computing device of the user. The property may be output to a display, such as electronic display coupled to the user's device. For instance, the property of the model may be provided at the GUI 314 of FIG. 3, as described above. The property of the model may additionally or alternatively be output to the data visualizer 318, which may perform additional analysis, as described herein. The process 400 may further involve outputting boundaries of different portions of the formation or the different values of a property of the formation over these different portions. Further, in some embodiments, the property of the model, boundaries, or different values of the property may be output to a data storage device.

FIG. 8 is a block diagram of an illustrative computer system 800 in which embodiments of the present disclosure may be implemented. For example, the functions, components, and/or operations of processing system 119, computing system 1040, or memory 121 of FIG. 1, system 300 of FIG. 3, process 400 of FIG. 4, and/or the process illustrated in FIG. 5, as described above, may be implemented using system 800. System 800 can be a computer, phone, PDA, or any other type of electronic device. Such an electronic device includes various types of computer readable media and interfaces for various other types of computer readable media. As shown in FIG. 8, system 800 includes a permanent storage device 802, a system memory 804, an output device interface 806, a system communications bus 808, a read-only memory (ROM) 810, processing unit(s) 812, an input device interface 814, and a network interface 816.

Bus 808 collectively represents all system, peripheral, and chipset buses that communicatively connect the numerous internal devices of system 800. For instance, bus 808 communicatively connects processing unit(s) 812 with ROM 810, system memory 804, and permanent storage device 802.

From these various memory units, processing unit(s) 812 retrieves instructions to execute and data to process in order to execute the processes of the subject disclosure. The processing unit(s) can be a single processor or a multi-core processor in different implementations.

ROM 810 stores static data and instructions that are needed by processing unit(s) 812 and other modules of system 800. Permanent storage device 802, on the other hand, is a read-and-write memory device. This device is a non-volatile memory unit that stores instructions and data even when system 800 is off. Some implementations of the subject disclosure use a mass-storage device (such as a magnetic or optical disk and its corresponding disk drive) as permanent storage device 802.

Other implementations use a removable storage device (such as a floppy disk, flash drive, and its corresponding disk drive) as permanent storage device 802. Like permanent storage device 802, system memory 804 is a read-and-write memory device. However, unlike storage device 802, system memory 804 is a volatile read-and-write memory, such a random access memory. System memory 804 stores some of the instructions and data that the processor needs at runtime. In some implementations, the processes of the subject disclosure are stored in system memory 804, permanent storage device 802, and/or ROM 810. For example, the various memory units include instructions for implementing the symbolic regression model, for training the symbolic regression model, and/or for estimating a property of the reservoir formation based on a model (e.g., mathematical expression) output by the symbolic regression model, in accordance with embodiments of the present disclosure, e.g., according to the symbolic regression and formation modeling operations performed by system 300 of FIG. 3 using process 400 of FIG. 4 and process 500 of FIG. 5, as described above. From these various memory units, processing unit(s) 812 retrieves instructions to execute and data to process in order to execute the processes of some implementations.

Bus 808 also connects to input and output device interfaces 814 and 806. Input device interface 814 enables the user to communicate information and select commands to the system 800. Input devices used with input device interface 814 include, for example, alphanumeric, QWERTY, or T9 keyboards, microphones, and pointing devices (also called “cursor control devices”). Output device interfaces 706 enables, for example, the display of images generated by the system 800. Output devices used with output device interface 806 include, for example, printers and display devices, such as cathode ray tubes (CRT) or liquid crystal displays (LCD). Some implementations include devices such as a touchscreen that functions as both input and output devices. It should be appreciated that embodiments of the present disclosure may be implemented using a computer including any of various types of input and output devices for enabling interaction with a user. Such interaction may include feedback to or from the user in different forms of sensory feedback including, but not limited to, visual feedback, auditory feedback, or tactile feedback. Further, input from the user can be received in any form including, but not limited to, acoustic, speech, or tactile input. Additionally, interaction with the user may include transmitting and receiving different types of information, e.g., in the form of documents, to and from the user via the above-described interfaces.

Also, as shown in FIG. 8, bus 808 also couples system 800 to a public or private network (not shown) or combination of networks through a network interface 816. Such a network may include, for example, a local area network (“LAN”), such as an Intranet, or a wide area network (“WAN”), such as the Internet. Any or all components of system 800 can be used in conjunction with the subject disclosure.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself. Accordingly, processes 400 and 500 of FIGS. 4 and 5, respectively, as described above, may be implemented using system 800 or any computer system having processing circuitry or a computer program product including instructions stored therein, which, when executed by at least one processor, causes the processor to perform functions relating to these methods.

As used in this specification and any claims of this application, the terms “computer”, “server”, “processor”, and “memory” all refer to electronic or other technological devices. These terms exclude people or groups of people. As used herein, the terms “computer readable medium” and “computer readable media” refer generally to tangible, physical, and non-transitory electronic storage mediums that store information in a form that is readable by a computer.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., a web page) to a client device (e.g., for purposes of displaying data to and receiving user input from a user interacting with the client device). Data generated at the client device (e.g., a result of the user interaction) can be received from the client device at the server.

It is understood that any specific order or hierarchy of steps in the processes disclosed is an illustration of exemplary approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Furthermore, the exemplary methodologies described herein may be implemented by a system including processing circuitry or a computer program product including instructions which, when executed by at least one processor, causes the processor to perform any of the methodology described herein.

A computer-implemented method of petrophysical modeling has been described. Embodiments of the method may include: receiving, by a computing device via a network from one or more data sources, training data for modeling a reservoir formation surrounding a wellbore drilled within the reservoir formation; training, by the computing device using symbolic regression, a machine learning model to determine a formation model representing the reservoir formation, based on the training data received from the one or more data sources; estimating, by the computing device, at least one property of the reservoir formation, based on the formation model; and performing a downhole operation along the wellbore within the reservoir formation, based on the at least one estimated property. Likewise, embodiments of a computer-readable storage medium having instructions stored therein have been described, where the instructions, when executed by a processor, may cause the processor to perform a plurality of functions, including functions to: receive, via a network from one or more data sources, training data for modeling a reservoir formation surrounding a wellbore drilled within the reservoir formation; train a machine learning model with symbolic regression to determine a formation model representing the reservoir formation, based on the received training data; and estimate at least one property of the reservoir formation, based on the formation model, wherein a downhole operation is performed along the wellbore within the reservoir formation, based on the at least one estimated property.

The foregoing embodiments of the method or computer-readable storage medium may include any one or any combination of the following elements, features, functions, or operations: the training data includes logging data received from a logging tool positioned within the wellbore and core sample data received from a core analysis tool, where the training comprises training the machine learning model to generate a plurality of formation models based on the logging data and the core sample data, and selecting one of the plurality of formation models, based on a predetermined fitness objective; ranking the plurality of formation models according to the predetermined fitness objective, and selecting one of the plurality of formation models based on the ranking; the predetermined fitness objective is defined by a fitness function based on a set of primitives representing measurement characteristics of the downhole tool and the core analysis tool; the training comprises generating a parent population of formation models, and performing crossover and mutation operations over a plurality of iterations until a predetermined termination condition is reached, wherein a child population of formation models is generated at each iteration based on the parent population generated at a preceding iteration, and wherein one of the plurality of formation models is selected from the child population of formation models generated from the crossover and mutation operations; at least one of the logging data or the core sample data includes NMR data, resistivity data, induction data, acoustic, density data, PE data, SP data, natural gamma ray data, and neutron data; the core analysis tool comprises at least one of permeameter, a porosimeter, or an imaging device; the at least one property is selected from the group consisting of an electrical efficiency parameter of reservoir rock associated with the reservoir formation, a tortuosity of reservoir rock associated with the reservoir formation, and a cementation of reservoir rock associated with the subsurface reservoir formation; the at least one property of the reservoir formation is selected from the group consisting of porosity, permeability, capillary pressure, bound fluid volume, shale volume, rock saturation, productivity index, relative permeability, effective permeability, hydrocarbon properties, and formation salinity; values of the at least one property are estimated for different portions of the reservoir formation based on the formation model, and based on the estimated values, determining a variation of the at least one property over the different portions of the reservoir formation, identifying boundaries of the different portions based on the variation of the values of the at least one property, and determining different rock facies of the reservoir formation, based on the identified boundaries; the formation model is represented by a target function; the target function is based on an Archie equation; and the target function is derived directly from the training data without using a predefined base function.

Furthermore, embodiments of a system including at least one processor and a memory coupled to the processor have been described, where the memory stores instructions, which, when executed by a processor, may cause the processor to perform a plurality of functions, including functions to: receive, via a network from one or more data sources, training data for modeling a reservoir formation surrounding a wellbore drilled within the reservoir formation; train a machine learning model with symbolic regression to determine a formation model representing the reservoir formation, based on the received training data; and estimate at least one property of the reservoir formation, based on the formation model, wherein a downhole operation is performed along the wellbore within the reservoir formation, based on the at least one estimated property.

The foregoing embodiments of the system may include any one or any combination of the following elements, features, functions, or operations: the training data includes logging data received from a logging tool positioned within the wellbore and core sample data received from a core analysis tool, where the training comprises training the machine learning model to generate a plurality of formation models based on the logging data and the core sample data, and selecting one of the plurality of formation models, based on a predetermined fitness objective; ranking the plurality of formation models according to the predetermined fitness objective, and selecting one of the plurality of formation models based on the ranking; the predetermined fitness objective is defined by a fitness function based on a set of primitives representing measurement characteristics of the downhole tool and the core analysis tool; the training comprises generating a parent population of formation models, and performing crossover and mutation operations over a plurality of iterations until a predetermined termination condition is reached, wherein a child population of formation models is generated at each iteration based on the parent population generated at a preceding iteration, and wherein one of the plurality of formation models is selected from the child population of formation models generated from the crossover and mutation operations; at least one of the logging data or the core sample data includes NMR data, resistivity data, induction data, acoustic, density data, PE data, SP data, natural gamma ray data, and neutron data; the core analysis tool comprises at least one of permeameter, a porosimeter, or an imaging device; the at least one property is selected from the group consisting of an electrical efficiency parameter of reservoir rock associated with the reservoir formation, a tortuosity of reservoir rock associated with the reservoir formation, and a cementation of reservoir rock associated with the subsurface reservoir formation; the at least one property of the reservoir formation is selected from the group consisting of porosity, permeability, capillary pressure, bound fluid volume, shale volume, rock saturation, productivity index, relative permeability, effective permeability, hydrocarbon properties, and formation salinity; values of the at least one property are estimated for different portions of the reservoir formation based on the formation model, and based on the estimated values, determining a variation of the at least one property over the different portions of the reservoir formation, identifying boundaries of the different portions based on the variation of the values of the at least one property, and determining different rock facies of the reservoir formation, based on the identified boundaries; the formation model is represented by a target function; the target function is based on an Archie equation; and the target function is derived directly from the training data without using a predefined base function.

While specific details about the above embodiments have been described, the above hardware and software descriptions are intended merely as example embodiments and are not intended to limit the structure or implementation of the disclosed embodiments. For instance, although many other internal components of the system 800 are not shown, those of ordinary skill in the art will appreciate that such components and their interconnection are well known.

In addition, certain aspects of the disclosed embodiments, as outlined above, may be embodied in software that is executed using one or more processing units/components. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives, optical or magnetic disks, and the like, which may provide storage at any time for the software programming.

Additionally, the flowchart and block diagrams in the figures illustrate the architecture, functionality, and operation of possible implementations of systems, methods and computer program products according to various embodiments of the present disclosure. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The above specific example embodiments are not intended to limit the scope of the claims. The example embodiments may be modified by including, excluding, or combining one or more features or functions described in the disclosure.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, integers, steps, operations, elements, and/or components, but do not preclude the presence or addition of one or more other features, integers, steps, operations, elements, components, and/or groups thereof. The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the embodiments in the form disclosed. Many modifications and variations will be apparent to those of ordinary skill in the art without departing from the scope and spirit of the disclosure. The illustrative embodiments described herein are provided to explain the principles of the disclosure and the practical application thereof, and to enable others of ordinary skill in the art to understand that the disclosed embodiments may be modified as desired for a particular implementation or use. The scope of the claims is intended to broadly cover the disclosed embodiments and any such modification.

What is claimed is:

1. A computer-implemented method comprising:
receiving, by a computing device via a network from one or more data sources, training data for modeling a reservoir formation surrounding a wellbore drilled within the reservoir formation;
training, by the computing device using symbolic regression, a machine learning model to determine a formation model representing the reservoir formation, based on the training data received from the one or more data sources;
estimating, by the computing device, at least one property of the reservoir formation, based on the formation model; and
performing a downhole operation along the wellbore within the reservoir formation, based on the at least one estimated property.

2. The computer-implemented method of claim 1, wherein the training data includes logging data received from a logging tool positioned within the wellbore and core sample data received from a core analysis tool, and wherein the training comprises:
training the machine learning model to generate a plurality of formation models based on the logging data and the core sample data; and
selecting one of the plurality of formation models, based on a predetermined fitness objective.

3. The computer-implemented method of claim 2, wherein selecting one of the plurality of formation models comprises:
ranking the plurality of formation models according to the predetermined fitness objective; and
selecting one of the plurality of formation models, based on the ranking.

4. The computer-implemented method of claim 2, wherein the predetermined fitness objective is defined by a fitness function based on a set of primitives representing measurement characteristics of the downhole tool and the core analysis tool.

5. The computer-implemented method of claim 2,
wherein the training comprises:
generating a parent population of formation models; and
performing crossover and mutation operations over a plurality of iterations until a predetermined termination condition is reached, wherein a child population of formation models is generated at each iteration based on the parent population generated at a preceding iteration, and
wherein one of the plurality of formation models is selected from the child population of formation models generated from the crossover and mutation operations.

6. The computer-implemented method of claim 2, wherein at least one of the logging data or the core sample data includes NMR data, resistivity data, induction data, acoustic, density data, PE data, SP data, natural gamma ray data, and neutron data.

7. The computer-implemented method of claim 2, wherein the core analysis tool comprises at least one of permeameter, a porosimeter, or an imaging device.

8. The computer-implemented method of claim 1, wherein the at least one property is selected from the group consisting of: an electrical efficiency parameter of reservoir rock associated with the reservoir formation; a tortuosity of reservoir rock associated with the reservoir formation; and a cementation of reservoir rock associated with the subsurface reservoir formation.

9. The computer-implemented method of claim 1, wherein the at least one property of the reservoir formation is selected from the group consisting of porosity, permeability, capillary pressure, bound fluid volume, shale volume, rock saturation, productivity index, relative permeability, effective permeability, hydrocarbon properties, and formation salinity.

10. The computer-implemented method of claim 1, wherein values of the at least one property are estimated for different portions of the reservoir formation based on the formation model, and the method further comprises:
determining, based on the estimated values, a variation of the at least one property over the different portions of the reservoir formation;
identifying boundaries of the different portions based on the variation of the values of the at least one property; and
determining different rock facies of the reservoir formation, based on the identified boundaries.

11. The computer-implemented method of claim 1, wherein the formation model is represented by a target function.

12. The computer-implemented method of claim 11, wherein the target function is based on an Archie equation.

13. The computer-implemented method of claim 11, wherein the target function is derived directly from the training data without using a predefined base function.

14. A system comprising:
a processor; and
a memory coupled to the processor having instructions stored therein, which when executed by the processor, cause the processor to perform a plurality of operations, including operations to:
receive, via a network from one or more data sources, training data for modeling a reservoir formation surrounding a wellbore drilled within the reservoir formation;
train a machine learning model with symbolic regression to determine a formation model representing the reservoir formation, based on the received training data; and
estimate at least one property of the reservoir formation, based on the formation model, wherein a downhole operation is performed along the wellbore within the reservoir formation, based on the at least one estimated property.

15. The system of claim 14, wherein the training data includes logging data received from a logging tool positioned within the wellbore and core sample data received from a core analysis tool, and wherein the operations performed by the processor include operations to:
train the machine learning model to generate a plurality of formation models based on the logging data and the core sample data; and
select one of the plurality of formation models, based on a predetermined fitness objective, wherein the predetermined fitness objective is defined by a fitness function based on a set of primitives representing measurement characteristics of the downhole tool and the core analysis tool.

16. The system of claim 15, wherein the operations performed by the processor include operations to:
rank the plurality of formation models according to the predetermined fitness objective; and
select one of the plurality of formation models, based on the ranking.

17. The system of claim 15, wherein the operations performed by the processor include operations to:
generate a parent population of formation models; and
perform crossover and mutation operations over a plurality of iterations until a predetermined termination condition is reached, wherein a child population of formation models is generated at each iteration based on the parent population generated at a preceding iteration, wherein one of the plurality of formation models is selected from the child population of formation models generated from the crossover and mutation operations.

18. The system of claim 14, wherein values of the at least one property are estimated for different portions of the reservoir formation based on the formation model, and the operations performed by the processor include operations to:
determine, based on the estimated values, a variation of the at least one property over the different portions of the reservoir formation; and
identify boundaries of the different portions based on the variation of the values of the at least one property; and
determine different rock facies of the reservoir formation, based on the identified boundaries.

19. The system of claim 14, wherein the formation model is represented by a target function, and the target function is derived from a predefined base function or directly from the training data without using the predefined base function.

20. A computer-readable storage medium having instructions stored thereon, which, when executed by a computer, cause the computer to perform a plurality of operations, including operations to:
receive, via a network from one or more data sources, training data for modeling a reservoir formation surrounding a wellbore drilled within the reservoir formation;
train a machine learning model with symbolic regression to determine a formation model representing the reservoir formation, based on the received training data; and
estimate at least one property of the reservoir formation, based on the formation model, wherein a downhole operation is performed along the wellbore within the reservoir formation, based on the at least one estimated property.
